(19) United States
(12) Patent Application Publication
(10) Pub. No.: US 2018/0003708 A1
(43) Pub. Date: Jan. 4, 2018

(54) DETECTION TARGET SENSING METHOD

(71) Applicant: KYOCERA CORPORATION, Kyoto (JP)

(72) Inventors: Kazuhiro NISHIZONO, Kizugawa-shi, Kyoto (JP); Hideharu KURIOKA, Kizugawa-shi, Kyoto (JP); Hiroyasu TANAKA, Kizugawa-shi, Kyoto (JP); Hiroshi KATTA, Kashihara-shi, Nara (JP); Atsuomi FUKUURA, Seika-cho, Soraku-gun (JP)

(21) Appl. No.: 15/547,466

(22) PCT Filed: Jan. 28, 2016
(86) PCT No.: PCT/JP2016/052478

§ 371 Date: Jul. 28, 2017

(30) Foreign Application Priority Data
Jan. 30, 2015 (JP) 2015-016650

(51) Int. Cl. G01N 33/553

(52) U.S. Cl. CPC G01N 33/553

(57) ABSTRACT

There is provided a sensing method which can detect a detection target with good sensitivity. A detection target sensing method includes supplying a detection target to a base having a first substance immobilized on a surface thereof, the detection target being bindable to the first substance; supplying a second substance to the base after the detection target is supplied thereto, the second substance being bindable to the detection target; and supplying a metal particle to the base after the second substance is supplied thereto, the metal particle being bindable to the second substance.

### TECHNICAL FIELD

The present invention relates to a sensing method for a detection target contained in an analyte.

### BACKGROUND ART

There is a heretofore known method for analyzing a target analyte contained in a sample with use of a detecting element such as a surface acoustic wave device (refer to Patent Literature 1, for example).

According to this analytical method, first, a first molecular recognition component, a nanoparticle-bound second molecular recognition component, and a target analyte contained in a sample are reacted with one another. After that, with the addition of a predetermined metal ion and a reducing agent, the metal ion is reduced to cause metal deposition for detection of a nanoparticle with deposited metal.

### CITATION LIST

### Patent Literature

Patent Literature 1: Japanese Unexamined Patent

Publication JP-A 2010-529422

### SUMMARY OF INVENTION

### Technical Problem

However, according to the technology described in Patent Literature as given above, the second molecular recognition component is supplied in a state of being kept bound to a nanoparticle, or equivalently in a combined-substance state, and, as compared with the component, as well as the nanoparticle, provided as a separate substance, the component-nanoparticle combined substance has a large and complex three-dimensional conformation. Consequently, when binding the above-described combined substance to a target analyte bound to the first molecular recognition component, the possibility arises that, due to steric hindrance entailed by the above-described three-dimensional conformation of the combined substance, the binding cannot be achieved properly. This makes it difficult to detect the target analyte contained in the sample with good sensitivity.

Thus, there has been a demand for a sensing method which can detect a detection target with good sensitivity.

### Solution to Problem

A detection target sensing method in accordance with an embodiment of the invention comprises: supplying a detection target to a base having a first substance immobilized on a surface thereof, the detection target being bindable to the first substance; supplying a second substance to the base after the detection target is supplied thereto, the second substance being bindable to the detection target; and supplying a metal particle to the base after the second substance is supplied thereto, the metal particle being bindable to the second substance.

### Advantageous Effects of Invention

According to the detection target sensing method in accordance with the embodiment of the invention, after supplying the detection target to the base having the first substance immobilized on the surface thereof, the base is supplied with the second substance which is bindable to the detection target, and hence, with the detection target kept bound efficiently to the first substance, the second substance can be bound efficiently to the detection target. Under this condition, further binding of the metal particle to the second substance makes possible detection of the detection target with better sensitivity.

### BRIEF DESCRIPTION OF DRAWINGS

FIG. 1 is views showing a sensor apparatus in accordance with an embodiment of the invention, wherein FIG. 1(a) is a plan view of the sensor apparatus, FIG. 1(b) is a lengthwise sectional view of the sensor apparatus taken along the line A-A of FIG. 1(a), and FIG. 1(c) is a widthwise sectional view of the sensor apparatus, illustrating, in top-to-bottom order as seen in the drawing on paper, the section taken along the line a-a of FIG. 1(a), the section taken along the line b-b thereof, and the section taken along the line c-c thereof;

FIG. 2 is enlarged sectional views showing part of the sensor apparatus shown in FIG. 1;

FIG. 3 is views showing a detecting element provided in the sensor apparatus shown in FIG. 1, wherein FIG. 3(a) is a plan view of the detecting element, FIG. 3(b) is a sectional view of the detecting element taken along the line d-d of FIG. 3(a), and FIG. 3(c) is a sectional view of the detecting element taken along the line e-e of FIG. 3(a);

FIG. 4 is an exploded plan view of the sensor apparatus shown in FIG. 1;

FIG. 5 is plan views showing manufacturing steps of the sensor apparatus shown in FIG. 1;

FIG. 6 is explanatory views of a detection target sensing method in accordance with an embodiment of the invention;

FIG. 7 is explanatory views of a wash solution supply step in the detection target sensing method in accordance with the embodiment of the invention; and

FIG. 8 is views showing modified examples of the detection target sensing method illustrated in FIG. 6.

### DESCRIPTION OF EMBODIMENTS

Hereinafter, a sensor apparatus in accordance with an embodiment of the invention will be detailed with respect to a case where an analyte has a liquid form (analyte liquid) with reference to drawings. In each of the drawings to be referred to in the following description, like constituent members are identified with the same reference symbols. Moreover, for example, the size of each member and the distance between the individual members are schematically shown in each drawing and may therefore differ from the actual measurements.

<Sensor Apparatus>

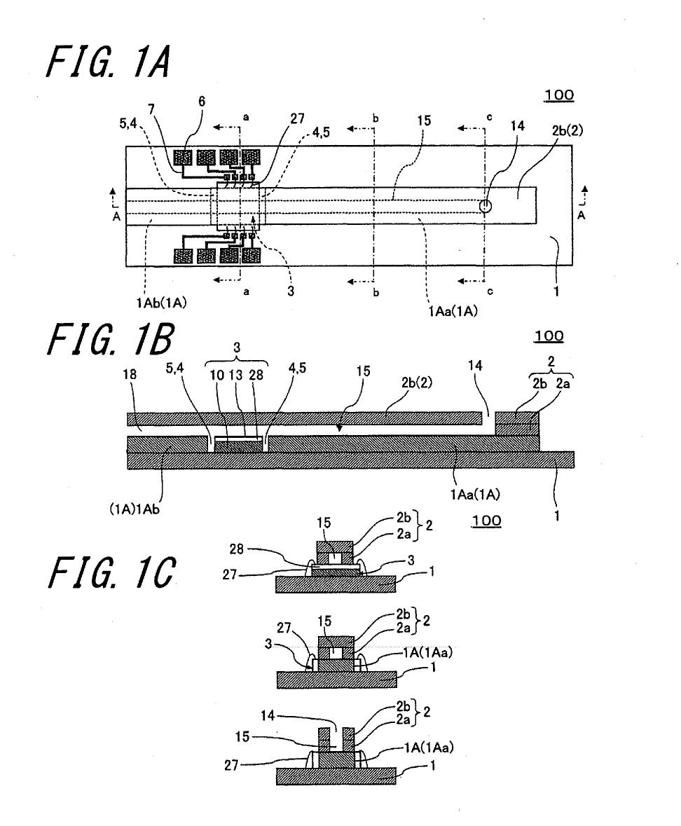

A sensor apparatus 100 in accordance with an embodiment of the invention will be described with reference to FIGS. 1 to 5.

As shown in FIG. 1, the sensor apparatus 100 according to the embodiment mainly comprises a first cover member 1, an intermediate cover member 1A, a second cover member 2, and a detecting element 3.

Specifically, as shown in FIG. 1(b), the sensor apparatus 100 has an inlet port 14 for admission of an analyte liquid, and a flow channel 15 which is continuous with the inlet port 14, is surrounded by the intermediate cover member 1A and the second cover member 2, and extends at least to a detecting section 13 of the detecting element 3. As shown in FIG. 1(b), the inlet port 14 is formed so as to pass through the second cover member 2 in a thickness direction thereof. Note that the inlet port 14 may be located on an upper face of the intermediate cover member 1A, as well as on a side face of the second cover member 2.

In the sensor apparatus 100 according to the embodiment, the detecting element 3 and the intermediate cover member 1A which constitutes at least part of the flow channel 15 are juxtaposed on an upper face of the first cover member 1, and therefore, even when using the detecting element 3 having a certain thickness, it is possible to leave the analyte liquid flow channel 15 extending from the inlet port 14 to the detecting section 13, and thereby allow an analyte liquid wicked through the inlet port 14 under capillarity or otherwise to flow to the detecting section 13. For example, the flow channel 15 has a width of 0.5 mm to 3 mm, and a depth of 0.1 mm to 0.5 mm. Thus, there can be provided the sensor apparatus 100 which, while employing the detecting element 3 having a certain thickness, has an analyte liquid suction mechanism built in itself, and affords simplicity in measurement operation. In a case where the sensor apparatus 100 is not provided with an analyte liquid suction mechanism of its own, admission of an analyte liquid can be accomplished by an instrument such as a pipette.

(First Cover Member 1)

As shown in FIG. 1(b), the first cover member 1 is shaped in a flat plate. The first cover member 1 has a thickness of 0.1 mm to 0.5 mm, for example. The first cover member 1 has substantially a rectangular planar configuration. The first cover member 1 has a longitudinal length of 1 cm to 5 cm, for example, and has a widthwise length of 1 cm to 3 cm, for example. As the material of the first cover member 1, it is possible to use, for example, paper, plastics, celluloid, ceramics, non-woven fabric, and glass. The use of plastics is desirable from the standpoints of required strength and cost.

Moreover, as shown in FIG. 1(a), a terminal 6 and a wiring line 7 routed from the terminal 6 to a location near the detecting element 3 are formed on the upper face of the first cover member 1. The terminal 6 is formed on the upper face of the first cover member 1 so as to lie on either side of the detecting element 3 in a width direction thereof. When measurement is made on the sensor apparatus 100 with an external measuring apparatus (not shown in the drawing), the terminal 6 and the external measuring apparatus are electrically connected to each other. Moreover, the terminal 6 and the detecting element 3 are electrically connected to each other via the wiring line 7 and so forth. A signal from the external measuring apparatus is inputted to the sensor apparatus 100 via the terminal 6, and, a signal from the sensor apparatus 100 is outputted to the external measuring apparatus via the terminal 6.

(Intermediate Cover Member 1A)

In this embodiment, as shown in FIGS. 1(a) and 1(b), the intermediate cover member 1A is placed in juxtaposition to the detecting element 3 on the upper face of the first cover member 1. Moreover, the intermediate cover member 1A and the detecting element 3 are located via an air gap.

The intermediate cover member 1A is a flat plate member having a recess-forming area 4, and a thickness thereof falls in the range of 0.1 mm to 0.5 mm, for example. As shown in FIG. 1, the intermediate cover member 1A may be made larger in thickness than the detecting element 3.

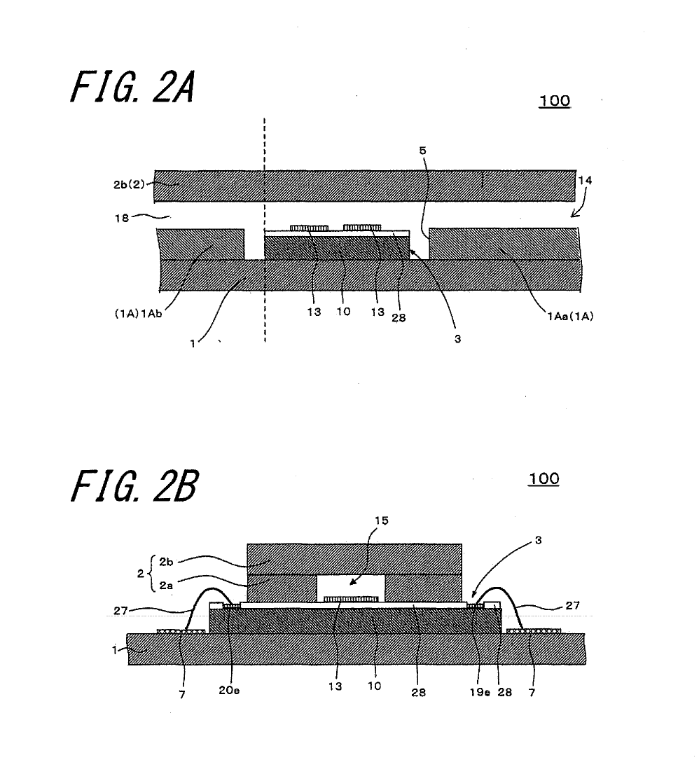

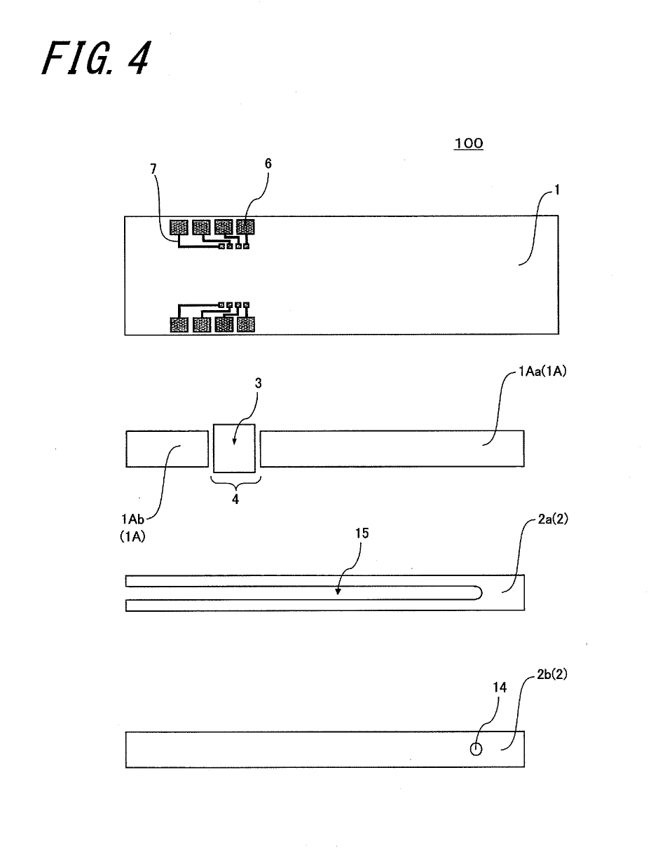

In this embodiment, as shown in FIGS. 1(a), 1(b), and 4, the recess-forming area 4 serves to divide the intermediate cover member 1A into a first upstream portion 1Aa and a first downstream portion 1Ab. The intermediate cover member 1A formed with the recess-forming area 4 is joined to the flat plate-shaped first cover member 1, whereupon an element receiving recess 5 is defined by the first cover member 1 and the intermediate cover member 1A as shown in FIG. 2(a). That is, the upper face of the first cover member 1 located inside the recess-forming area 4 becomes a bottom face of the element receiving recess 5, and an inner wall of the recess-forming area 4 becomes an inner wall of the element receiving recess 5. In other words, a part of the upper face of the first cover member 1 which is exposed from the recess-forming area 4 becomes the bottom face of the element receiving recess 5, and the inner wall of the recess-forming area 4 becomes the inner wall of the element receiving recess 5.

As the material of the intermediate cover member 1A, it is possible to use, for example, resin (including plastics), paper, non-woven fabric, and glass. More specifically, resin materials such as polyester resin, polyethylene resin, acrylic resin, and silicone resin can be used. The first cover member 1 and the intermediate cover member 1A may be formed either of the same material or of different materials.

Moreover, in this embodiment, the intermediate cover member 1A comprises the first upstream portion 1Aa and the first downstream portion 1Ab, and, as shown in FIG. 1(a), in the sensor apparatus 100 as viewed transparently from above an upper face of the second cover member 2 (as seen in a top transparent plan view), the detecting element 3 is located between the first upstream portion 1Aa and the first downstream portion 1Ab. With this arrangement, when an analyte liquid flows out over the detecting element 3 after passing through that part of the flow channel 15 which corresponds to the first upstream portion 1Aa, an excess of the analyte liquid over that required for measurement flows toward the first downstream portion 1Ab, whereby an adequate amount of the analyte liquid can be fed to the detecting element 3.

(Second Cover Member 2)

As shown in FIGS. 1(b) and 1(c), the second cover member 2 covers at least part of the detecting element 3, and is joined to the intermediate cover member 1A. As the material of the second cover member 2, it is possible to use, for example, resin (including plastics), paper, non-woven fabric, and glass. More specifically, resin materials such as polyester resin, polyethylene resin, acrylic resin, and silicone resin can be used. It is advisable that the second cover member 2 and the first cover member 1 are formed of the same material. This makes it possible to reduce deformation resulting from the difference in thermal expansion coefficient between these cover members. The second cover member 2 may either be joined only to the intermediate cover member 1A or be joined to both of the first cover member 1 and the intermediate cover member 1A.

The second cover member 2 comprises a third substrate 2a and a fourth substrate 2b.

The third substrate 2a is bonded to the upper face of the intermediate cover member 1A. The third substrate 2a is shaped like a flat plate having a thickness of 0.1 mm to 0.5 mm, for example. The fourth substrate 2b is bonded to an upper face of the third substrate 2a. The fourth substrate 2b is shaped like a flat plate having a thickness of 0.1 mm to 0.5 mm, for example. As shown in FIG. 4, the third substrate 2a is provided with a cutaway to constitute the flow channel 15, and, when joining the fourth substrate 2b to the third substrate 2a, as shown in FIG. 1(b), the flow channel 15 is formed on a lower face of the fourth substrate 2b. The flow channel 15 extends from the inlet port 14 to at least a region immediately above the detecting section 13, and, as shown in FIG. 1(c), the section of the flow channel 15 perpendicular to an extension direction of the flow channel 15 has a rectangular profile, for example.

In this embodiment, as shown in FIG. 1(b), the third substrate 2a is not present at a downstream end of the flow channel 15, and, a gap between the fourth substrate 2b and the intermediate cover member 1A serves as an air release hole 18. The air release hole 18 serves to let air and so forth present in the flow channel 15 go out.

(Detecting Element 3)

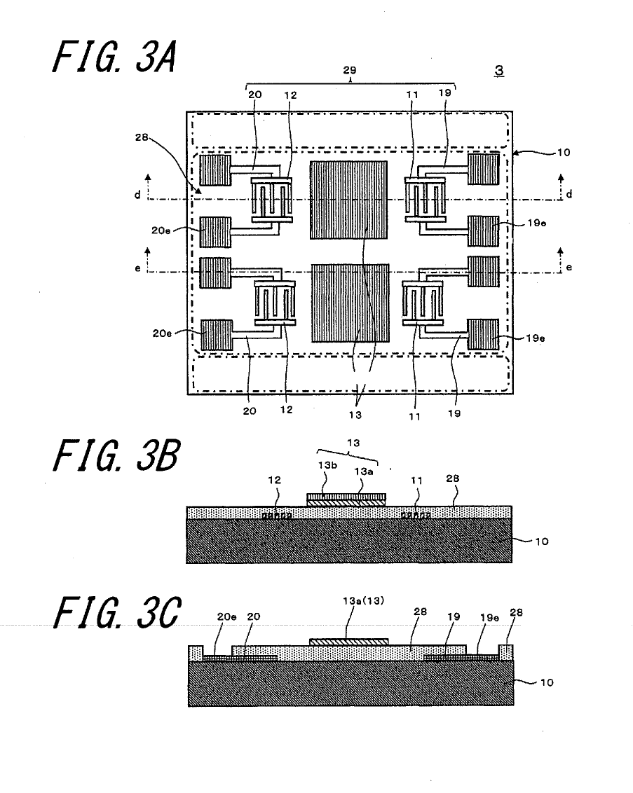

As shown in FIG. 1(b), the detecting element 3 comprises: an element substrate 10 located on the upper face of the first cover member 1; and at least one detecting section 13 located on an upper face of the element substrate 10 or on an upper face of an insulating member 28 which will hereafter be described, for detecting a detection target 13c contained in an analyte liquid. The details of the detecting element 3 are shown in FIG. 2(b) and FIG. 3.

In this embodiment, as shown in FIG. 3, on the upper face of the element substrate 10, there is provided an element electrode (electrode pattern) 29, and also an insulating member 28 is provided so as to cover the element electrode 29. When employing a SAW element as the detecting element 3, the element electrode 29 corresponds to an IDT (Interdigital Transducer) electrode, an extraction electrode, and so forth. In this embodiment, as shown in FIG. 3, on the upper face of the element substrate 10, there are arranged a first IDT electrode 11, a second IDT electrode 12, a first extraction electrode 19, a second extraction electrode 20, and so forth that will hereafter be described.

In this embodiment, as shown in FIG. 2(b),for example, the second cover member 2 is fixedly disposed above the IDT electrodes 11 and 12 on an upper face of the detecting element 3.

(Element Substrate 10)

The element substrate 10 is composed of a substrate of single crystal having piezoelectric properties, such for example as lithium tantalate (LiTaO₃) single crystal, lithium niobate (LiNbO₃) single crystal, or quartz. The planar configuration and dimensions of the element substrate 10 may be suitably determined. By way of example, the element substrate 10 has a thickness of 0.3 mm to 1 mm.

The following describes, as the element electrode 29, the IDT electrode 11 and 12, and the extraction electrodes 19 and 20 in the order named.

(IDT Electrodes 11 and 12)

As shown in FIG. 3, the first IDT electrode 11 is located on the upper face of the element substrate 10, and comprises a pair of comb electrodes. Each comb electrode includes two bus bars opposed to each other, and a plurality of electrode fingers each extending from corresponding one of the bus bars toward the other. The comb electrode pair is disposed so that a plurality of the electrode fingers are arranged in an interdigitated pattern. As with the first IDT electrode 11, the second IDT electrode 12 is also located on the upper face of the element substrate 10, and comprises a pair of comb electrodes. The first IDT electrode 11 and the second IDT electrode 12 as shown in FIG. 3 constitute a transversal IDT electrode.

The first IDT electrode 11 is intended for generation of a predetermined surface acoustic wave (SAW), and the second IDT electrode 12 is intended for reception of the SAW generated in the first IDT electrode 11. Hence, the first IDT electrode 11 and the second IDT electrode 12 are positioned on the same straight line so that the SAW generated in the first IDT electrode 11 can be received by the second IDT electrode 12. The design of frequency response characteristics of SAW can be made on the basis of the number of the electrode fingers of the first IDT electrode 11 and the second IDT electrode 12, the distance between the adjacent electrode fingers, the crossing width of the electrode fingers, etc., used as parameters. Among various vibration modes for SAW to be excited by the IDT electrode, for example, a vibration mode of transversal waves called SH waves (shear horizontal waves) is utilized in the detecting element 3 according to the embodiment.

For example, the frequency of SAW may be set within a range of several megahertz (MHz) to several gigahertz (GHz). By setting the SAW frequency within the range of a several hundred MHz to several GHz in particular, it is possible to provide suitability for practical use, and also to achieve a reduction in size of the detecting element 3 that will eventually lead to miniaturization of the sensor apparatus 100.

As the material of the first IDT electrode 11 and the second IDT electrode 12, it is possible to use, for example, gold, aluminum, or an alloy of aluminum and copper (aluminum alloy). Moreover, these electrodes may be designed to have a multilayer structure. When adopting a multilayer structure, for example, the electrode may be composed of the first layer containing titanium or chromium, the second layer containing gold, aluminum, or an aluminum alloy, and the third layer containing titanium or chromium. In this case, it is advisable to subject titanium or chromium constituting the third layer to surface oxidation for enhancement in adherability between the electrode and SiO₂ constituting the insulating member 28 which will hereafter be described. Specific examples of the multilayer structure include a three-layer structure obtained by successively forming a gold layer and a titanium layer in the order named on a titanium layer (Ti/Au/Ti) and a three-layer structure obtained by successively forming a gold layer and a titanium oxide layer in the order named on a titanium layer (Ti/Au/TiO₂). Moreover, when adopting a multilayer structure, the uppermost one of a plurality of layers constituting the multilayer structure may be formed of a material which differs from that used for an immobilization film 13a which will hereafter be described. This holds true for the first extraction electrode 19 and the second extraction electrode 20 that will hereafter be described.

Moreover, the first IDT electrode 11 and the second IDT electrode 12 may be designed to have a thickness of 30 nm to 300 nm, for example. In a case where the first IDT electrode 11 and the second IDT electrode 12 have a thickness of greater than or equal to 30 nm, transmission loss of surface acoustic waves can be reduced. On the other hand, in a case where the first IDT electrode 11 and the second IDT electrode 12 have a thickness of less than or equal to 300 nm, a deterioration in detection sensitivity can be reduced.

(Extraction Electrodes 19 and 20)

As shown in FIG. 3(a), the first extraction electrode 19 is connected to the first IDT electrode 11, and the second extraction electrode 20 is connected to the second IDT electrode 12.

Moreover, the first extraction electrode 19 is extracted from the first IDT electrode 11 in the opposite direction to the detecting section 13, and, an end 19e of the first extraction electrode 19 is electrically connected to the wiring line 7 disposed in the first cover member 1. The second extraction electrode 20 is extracted from the second IDT electrode 12 in the opposite direction to the detecting section 13, and, an end 20e of the second extraction electrode 20 is electrically connected to the wiring line 7. As shown in FIGS. 3(a) and 3(c), the end 19e of the first extraction electrode 19 and the end 20e of the second extraction electrode 20 are exposed without being covered with the insulating member 28 which will hereafter be described. In FIG. 3(a), the exposed components left uncovered with the insulating member 28 are patterned (shaded) with longitudinal lines.

As the material of the first extraction electrode 19 and the second extraction electrode 20, it is possible to use a material similar to that used for the first IDT electrode 11 and the second IDT electrode 12. Moreover, in a case where the end 19e of the first extraction electrode 19 and the end 20e of the second extraction electrode 20 have a multilayer structure, specific examples of the multilayer structure include a two-layer structure in which a gold layer is formed on a titanium layer (Ti/Au), a five-layer structure in which a gold layer, a titanium layer, a titanium layer, and a gold layer are successively formed in the order named on a titanium layer (Ti/Au/Ti/Ti/Au), and a five-layer structure in which a gold layer, a titanium oxide layer, a titanium layer, and a gold layer are successively formed in the order named on a titanium layer (Ti/Au/TiO₂/Ti/Au).

The first extraction electrode 19 and the second extraction electrode 20 may be designed to have a thickness of 30 nm to 300 nm, for example. This makes it possible to ensure energization between the first IDT electrode 11 and the second IDT electrode 12. Moreover, the first extraction electrode 19 and the second extraction electrode 20 may be made equal in thickness to the first IDT electrode 11 and the second IDT electrode 12. This makes it possible to produce the extraction electrode and the IDT electrode in one step, and thereby simplify the manufacturing process, and also to avoid formation of a stepped electrode surface at the juncture between the extraction electrode and the IDT electrode, and thereby attain uniformity in adhesion with the insulating member 28. In consequence, for example, it is possible to suppress cracking caused in the insulating member 28 by stress application.

(Insulating Member 28)

The insulating member 28, which is conducive to, for example, prevention of oxidation in the element electrode (the IDT electrodes 11 and 12, the extraction electrodes 19 and 20, etc.) 29, covers at least part of the element electrode 29 as shown in FIG. 3.

In this embodiment, as shown in FIG. 3(b), the insulating member 28 covers the first IDT electrode 11 and the second IDT electrode 12. Moreover, the insulating member 28 also covers the first extraction electrode 19 and the second extraction electrode 20. However, as shown in FIGS. 3(a) and 3(c), in each of the end 19e of the first extraction electrode 19 and the end 20e of the second extraction electrode 20, at least a part thereof is uncovered with the insulating member 28. As shown in FIG. 2(b), this uncovered part and the wiring line 7 are electrically connected to each other via a metallic thin wire (lead wire) 27. Note that the insulating member 28 may be formed so as to cover the metallic thin wire 27 and the wiring line 7.

Examples of the material of the insulating member 28 include silicon oxide (SiO₂), aluminum oxide, zinc oxide, titanium oxide, silicon nitride, and silicon.

Moreover, the insulating member 28 may be designed to have a thickness of 10 nm to 2000 nm, for example. In a case where the insulating member 28 has a thickness of greater than or equal to 10 nm, it is possible to attain excellent temperature characteristics, and also to provide sufficient insulation from the IDT electrodes 11 and 12 and so forth. On the other hand, in a case where the insulating member 28 has a thickness of less than or equal to 2000 nm, it is possible to reduce a deterioration in detection sensitivity, and also to attain excellent temperature characteristics.

(Detecting Section 13)

As shown in FIG. 3, the detecting section 13, which detects a detection target 13c contained in an analyte liquid, is located on the upper face (surface) of the element substrate 10 or on the upper face of the insulating member 28 so as to lie between the first IDT electrode 11 and the second IDT electrode 12.

In this embodiment, the detecting section 13 comprises: an immobilization film 13a located on the upper face (surface) of the element substrate 10 or on the upper face (surface) of the insulating member 28; and a reaction portion 13b located on an upper face of the immobilization film 13a. By way of another example, the immobilization film 13a may be omitted from the detecting section 13, and, in this case, the reaction portion 13b is located on the upper face (surface) of the element substrate 10 or on the upper face (surface) of the insulating member 28.

(Immobilization Film 13a)

The immobilization film 13a is located on the upper face (surface) of the element substrate 10 or on the upper face (surface) of the insulating member 28, and serves to immobilize the reaction portion 13b at an upper face (surface) thereof. In this embodiment, as described above, since the detecting section 13 is located between the first IDT electrode 11 and the second IDT electrode 12, it follows that the immobilization film 13a is also located between the first IDT electrode 11 and the second IDT electrode 12.

As the material of the immobilization film 13a, it is possible to use, for example, a metal, an oxide film (Such as SiO₂ film or TiO₂ film), and a polymer film (Such as PET film or PMMA film). When using a metal for the immobilization film 13a, or when imparting a multilayer structure to the immobilization film 13a, an outer surface (outermost layer) of the multilayer structure may be formed of an oxide film and a polymer film as described just above. The immobilization film 13a may be composed of the same material as that used for the element electrode 29, such as the first IDT electrode 11 and the second IDT electrode 12. Moreover, in addition to the same material as that used for the first IDT electrode 11 and the second IDT electrode 12, other noble metal materials (for example, platinum, silver, palladium, and an alloy of these metals) can be used as the material of the immobilization film 13a. Furthermore, when imparting a multilayer structure to the immobilization film 13a, for example, the multilayer structure may be of a two-layer structure consisting of a chromium or titanium layer and a gold layer formed on the chromium (titanium) layer, or a three-layer structure consisting of a chromium or titanium layer, a gold layer formed thereon, and a titanium oxide layer formed on the gold layer. Specific examples of the multilayer structure include a two-layer structure in which a gold layer is formed on a titanium layer (Ti/Au) and a three-layer structure in which a gold layer and a titanium oxide layer are successively formed in the order named on a titanium layer (Ti/Au/TiO₂).

The immobilization film 13a may be designed to have a thickness of 30 nm to 300 nm, for example. In a case where the immobilization film 13a has a thickness of greater than or equal to 30 nm, a deterioration in detection sensitivity can be reduced. On the other hand, in a case where the immobilization film 13a has a thickness of less than or equal to 300 nm, transmission loss of surface acoustic waves can be reduced.

(Reaction Portion 13b)

The reaction portion 13b, which undergoes chemical reaction with the detection target 13c contained in the analyte liquid, is located on the surface (upper face) of the immobilization film 13a as shown in FIG. 3. Examples of the reaction portion 13b include a structure in which a first substance 13b3 is immobilized on the surface of the immobilization film 13a via a functional group, and a structure in which the first substance 13b3 is immobilized on the surface of the immobilization film 13a via a functional group and an organic member. In the reaction portion 13b having such a structure, for example, upon contact with the analyte liquid, a predetermined detection target 13c contained in the analyte liquid is bound to the first substance 13b3, etc. corresponding to the detection target 13c, such as an aptamer.

While an example of the functional group is an SH functional group (thiol group), in addition to that, a silanol group, an amino group, a carboxyl group, a maleimide group, a sulfide group, a disulfide group, an aldehyde group, an azide group, an N-hydroxysuccinimide group, an epoxy group, a carbonyldiimidazole group, an isocyanate group, a hydroxyl group, a hydrazide group, a vinyl group, a tosyl group, a tresyl group, a succinimide group, a sulfonated succinimide group, and biotin may be given by way of example.

Examples of the organic member include dextran, agarose, alginic acid, carrageenan, saccharides of the kind just described, and derivatives of such a saccharide, polyvinyl alcohol, polyacrylamide, polyacrylic acid, oligoethylene glycol, polyethylene glycol, betaine polymer, cellulose, organic polymers of the kind just described, and a self-assembled monolayer (SAM). An example of the self-assembled monolayer is one containing a linear or branched hydrocarbon chain having a carbon length of about 1 to 400 carbons. The hydrocarbon chain may contain an alkyl group, an aryl group, an alkenyl group, an alkynyl group, a cycloalkyl group, an alkaryl group, an aralkyl group, or a combination of some of these groups. A self-assembled monolayer of HS—(CH₂)ₙ—NH³⁺Cl⁻ or HS—(CH₂)ₙ—COOH may be given as an example. In this case, an alkyl chain having a carbon length of about 3 to 30 carbons (represented by n) can be employed.

The first substance 13b3 is possessed of a molecular recognition capability for selective binding to a specific substance, and, examples of the first substance 13b3 include peptide, protein substances (including antibody, enzyme, and lectins), nucleic acid (including aptamer), and boronic acid compounds. Moreover, as described above, the first substance 13b3 can be immobilized on the surface of the immobilization film 13a via a functional group or via an organic member having a homobifunctional group or heterobifunctional group at each terminus. For example, it is advisable that the first substance 13b3 such as an aptamer is immobilized on an upper face (top) of an organic member covering substantially the entire area of the surface of the immobilization film 13a, or that the first substance 13b3 such as an aptamer is immobilized on the surface of the immobilization film 13a via a functional group, and then an organic member is immobilized around the immobilized aptamer. This makes it possible to immobilize the aptamer in an oriented position, and thereby achieve efficient immobilization of as large an amount as possible of the aptamer on the surface of the immobilization film 13a. That is, by binding a functional group to one terminus of the aptamer, it is possible to orient a detection target 13c-bound area at the other terminus of the aptamer in a direction from the immobilization film 13a upward, and thereby place individual aptamers adjacent one another in a close arrangement.

For example, the sensor apparatus 100 thus far described can be produced in the following manner.

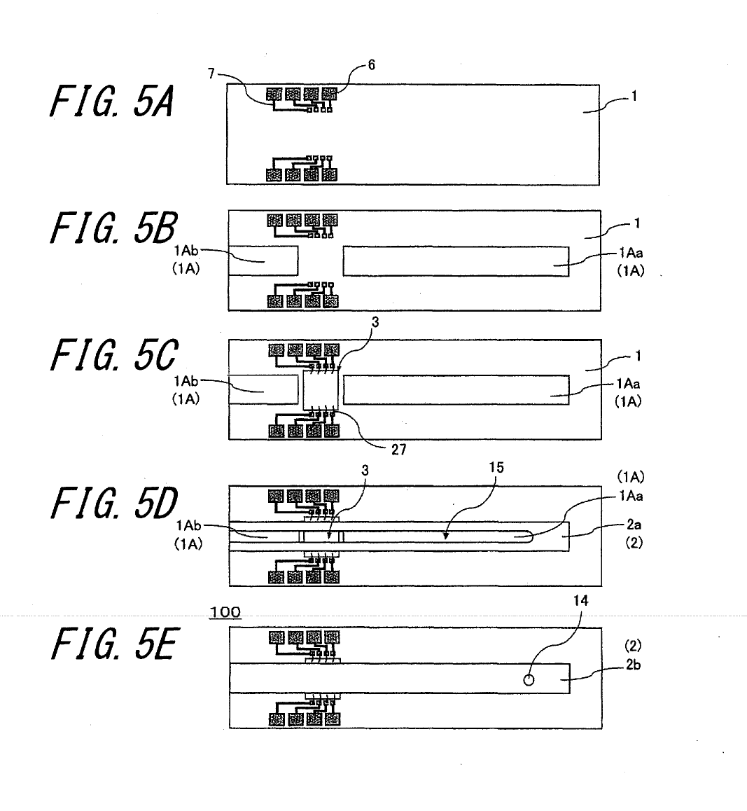

As shown in FIG. 5(a), first, the first cover member 1 provided with the terminals 6 and the wiring lines 7 is prepared.

Next, as shown in FIG. 5(b), the intermediate cover member 1A is laminated onto the first cover member 1. The intermediate cover member 1A is composed of the first upstream portion 1Aa and the first downstream portion 1Ab.

Next, as shown in FIG. 5(c), the detecting element 3 is mounted so as to lie between the first upstream portion 1Aa and the first downstream portion 1Ab of the intermediate cover member 1A via the metallic thin wire 27. Note that either of the intermediate cover member 1A and the detecting element 3 may be the first to be placed on the first cover member 1.

Next, as shown in FIG. 5(d), the third substrate 2a of the second cover member 2 is laminated onto the intermediate cover member 1A.

Then, as shown in FIG. 5(e), by laminating the fourth substrate 2b onto the third substrate 2a, the sensor apparatus 100 according to the embodiment is produced.

Moreover, in the course of production of the sensor apparatus 100 according to the embodiment, a procedure in the making of the detecting element 3 comprises the following steps (i) through (iv).

(i) a step of forming the first IDT electrode 11, the second IDT electrode 12, the first extraction electrode 19, and the second extraction electrode 20 by resist patterning with subsequent lifting-off operation.

(ii) a step of forming the insulating member 28 by film-forming process with subsequent patterning operation.

(iii) a step of forming the immobilization film 13a, the end 19e of the first extraction electrode 19, and the end 20e of the second extraction electrode 20.

(iv) a step of supplying a solution containing an organic member having a homobifunctional group or heterobifunctional group at each terminus to the immobilization film 13a, and subsequently supplying and immobilizing a solution containing the first substance 13b3.

<Detection Target Sensing Method>

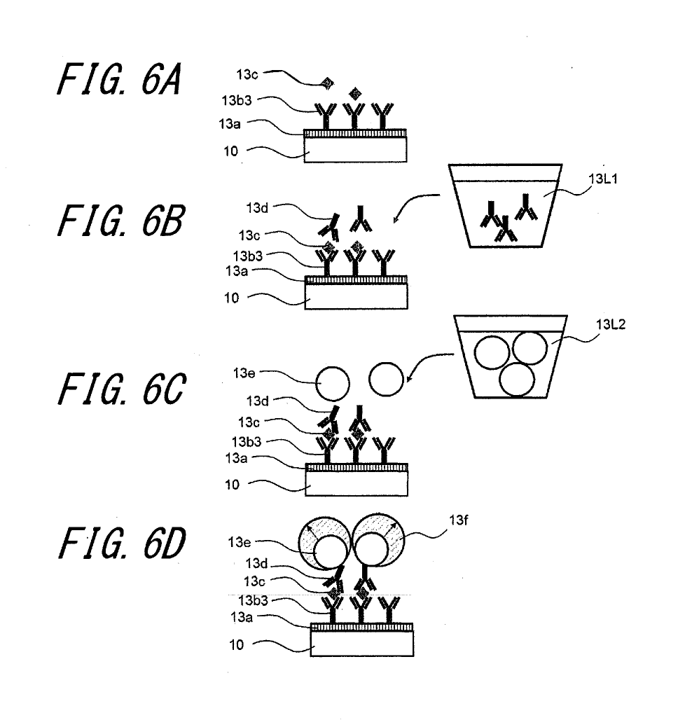

A detection target sensing method in accordance with an embodiment of the invention will be described with reference to FIGS. 6 to 8.

Specifically, the detection target sensing method according to the embodiment comprises: a step of supplying a detection target 13c to a base 10 having a first substance 13b3 immobilized on a surface thereof, the detection target 13c being bindable to the first substance 13b3; a step of supplying a second substance 13d to the base 10 after the detection target 13c is supplied thereto, the second substance 13d being bindable to the detection target 13c; and a step of supplying a metal particle 13e to the base 10 after the second substance 13d is supplied thereto, the metal particle 13e being bindable to the second substance 13d. In what follows, the element substrate 10 may be described as an example of the base 10.

(Step of Immobilizing First Substance 13b3 on Base 10 Surface)

As described above, first, the first substance 13b3 is immobilized on the surface of the base 10 via the immobilization film 13a, a functional group, an organic member, etc. The related particulars can be seen from the foregoing and will thus be omitted from the following description. The following describes a case where the first substance 13b3 is immobilized on the surface of the base 10 via the immobilization film 13a.

(Step of Supplying Detection Target 13c)

Next, as shown in FIG. 6(a), the base 10 having the first substance 13b3 immobilized on its surface is supplied with the detection target 13c which is bindable to the first substance 13b3. At this time, the detection target 13c may be supplied in a state of being contained in a predetermined analyte liquid.

Examples of the detection target 13c include a protein substance such as antibody, enzyme, or albumin, and also lipid, bacteria, virus, metabolite, and nucleic acid. Moreover, examples of the analyte liquid include blood, blood serum, blood plasma, urine, saliva, sweat, tears, and sputum that are each provided either in an as-is state or in the form of a dilute solution prepared by dilution with a suitable solvent.

In this step, the detection target 13c can be bound efficiently to the first substance 13b3 immobilized on the surface of the base 10.

(Step of Supplying Second Substance 13d)

Next, as shown in FIG. 6(b), after supplying the detection target 13c as described above, the base 10 is supplied with the second substance 13d which is bindable to the detection target 13c.

Like the first substance 13b3, the second substance 13d is also possessed of a molecular recognition capability for selective binding to a specific substance, and, examples of the second substance 13d include peptide, protein substances (including antibody, enzyme, and lectins), nucleic acid (including aptamer), and boronic acid compounds.

In this step, after supplying the detection target 13c, the second substance 13d is supplied separately at another time, and hence, the second substance 13d can be bound efficiently to the detection target 13c bound to the first substance 13b3 in the preceding step. That is, for example, in the case of supplying the second substance 13d in a state of being kept bound to the metal particle 13e which will hereafter be described, or equivalently in a combined-substance state, the binding of the second substance 13d to the detection target 13c bound to the first substance 13b3 could be impaired due to steric hindrance entailed by the dimensions of the second substance-metal particle combined substance in itself. In contrast, as described above, by supplying the second substance 13d alone without being bound to the metal particle 13e, it is possible to suppress steric hindrance as described above. This makes it possible to suppress an impairment of the binding of the second substance 13d to the detection target 13c, or reduce a decrease in the rate of binding reaction between the detection target 13c and the second substance 13d.

As shown in FIG. 6(b), the second substance 13d can be supplied in a state of being contained in a first solution 13L1 to the base 10. In this case, in contrast to a case where the second substance 13d is put in a predetermined solution together with the metal particle 13e, it is possible to select an optimum solution for the second substance 13d, and thereby, for example, restrain second substance 13d agglomeration, and also provide enhanced bindability between the second substance 13d and the detection target 13c, even if the second substance concentration is high. In consequence, even when the analyte liquid has a low content of the detection target 13c (contains the detection target 13c at low concentration), the binding of the second substance 13d to the detection target 13c makes possible detection with good sensitivity.

Examples of the first solution 13L1 include a phosphoric acid buffer solution, a citric acid buffer solution, a HEPES (4-(2-hydroxyethyl)-1-piperazineethanesulfonic acid) buffer solution, and a MOPS (3-Morpholinopropanesulfonic acid) búffer solution. Sodium chloride, potassium chloride, magnesium chloride, or EDTA (ethylenediaminetetraacetic acid) may be contained in such a solution. Moreover, on an as needed basis, a surfactant, such as Tween 20 (registered trademark) or Triton X100 (registered trademark), may be contained in the solution.

When adopting antibody for use as the second substance 13d, a phosphate buffered saline solution containing Tween 20 in appropriate concentrations can be used. Moreover, when adopting nucleic acid for use as the second substance 13d, a Tris (tris(hydroxymethyl)aminomethane) buffer solution containing 5 mM EDTA can be used.

(Step of Supplying First Wash Solution 13W1)

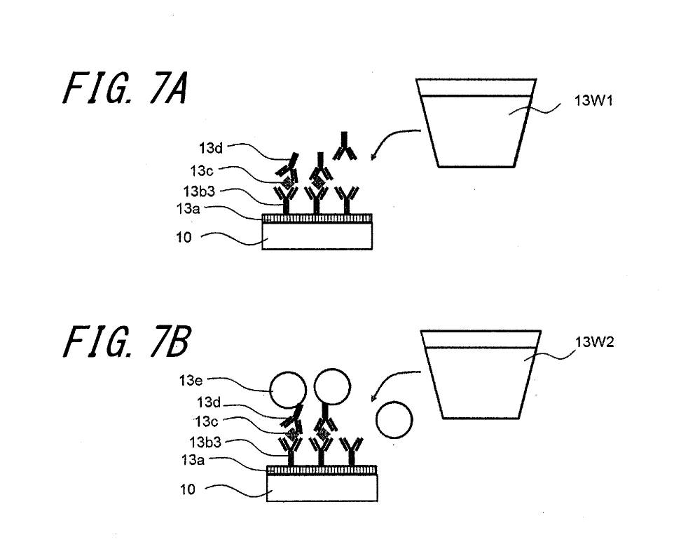

Next, as shown in FIG. 7(a), a first wash solution 13W1 may be supplied to the base 10 after supplying the second substance 13d and before supplying the metal particle 13e.

In this step, for example, the second substance 13d which remains unbound to the detection target 13c can be removed from the base 10 and vicinal areas. In consequence, in the subsequent step of supplying the metal particle 13e, it is possible to reduce the likelihood of the metal particle 13e being bound to an unnecessary residual substance, and thereby bind the metal particle 13e efficiently to the second substance 13d bound to the detection target 13c.

For example, the first wash solution 13W1 may either be identical with or differ from the first solution 13L1. In a case where the first wash solution 13W1 differs from the first solution 13L1, for example, the first wash solution 13W1 may be made larger in surfactant concentration than the first solution 13L1, or may be prepared by adding a different additional surfactant to the first solution 13L1. This makes it possible to efficiently remove the second substance 13d which remains unbound to the detection target 13c from the base 10.

(Step of Supplying Linker 13L)

Next, the base 10 may be supplied with a linker 13L which is bindable to the second substance 13d and the metal particle 13e. In this case, the linker 13L is supplied after supplying the second substance 13d and before supplying the metal particle 13e.

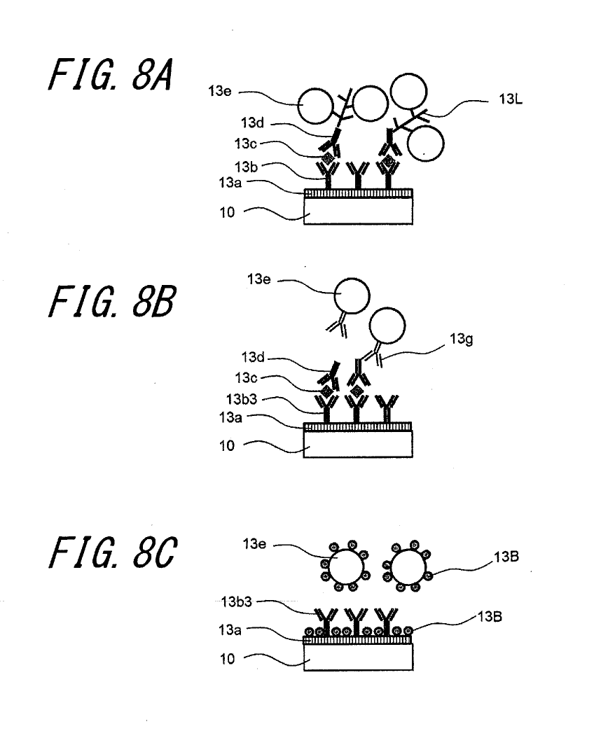

In this step, as shown in FIG. 8(a), for example, even with lack of desired bindability between the second substance 13d and the metal particle 13e, they can be bound to each other efficiently via the linker 13L. Meanwhile, even in a case where the second substance 13d and the metal particle 13e can be directly bound to each other, the use of a suitable linker 13L enables more efficient binding between them.

The linker 13L may be composed of a first linker which is bindable to the second substance 13d and a second linker which is bindable to the metal particle 13e. In this case, the second substance 13d can be supplied in a state of being kept bound to the first linker, whereafter the metal particle 13e can be supplied in a state of being kept bound to the second linker. In the alternative, after supplying the second substance 13d, the first linker, the second linker, and the metal particle 13e may be successively supplied in the order named in a state of not binding to each other. Thereby, the second substance 13d and the metal particle 13e can be bound to each other via the first linker and the second linker.

An example of the linker 13L is a combination of streptavidin and biotin. In addition to that, a combination of histidine-tag and Ni-NTA (nitrilotriacetate), a combination of DNA and complementary DNA, a combination of any lectin and sugar chain, a combination of cis-diol compound and boronic acid compound, Au-tag peptide, protein A, and protein G may be given by way of example.

As a specific example, with use of a combination of streptavidin and biotin, the second substance 13d and the metal particle 13e can be bound to each other via streptavidin bound to the second substance 13d and biotin bound to the metal particle 13e. In the alternative, the second substance 13d and the metal particle 13e may be bound to each other via biotin bound to the second substance 13d and streptavidin bound to the metal particle 13e. Moreover, in a case where the second substance 13d is antibody, with use of a combination of histidine-tag and Ni-NTA, the second substance 13d and the metal particle 13e can be bound to each other efficiently via histidine-tag added to the second substance 13d and Ni-NTA bound to the metal particle 13e.

(Step of Supplying Metal Particle 13e)

Next, as shown in FIG. 6(c), after supplying the second substance 13d, the base 10 is supplied with the metal particle 13e which is bindable to the second substance 13d. Examples of the metal particle 13e include gold and platinum.

In this step, after supplying the second substance 13d, the metal particle 13e is supplied separately at another time, and hence, the metal particle 13e can be bound efficiently to the second substance 13d bound to the detection target 13c in the preceding step. That is, as described above, by supplying the metal particle 13e alone, it is possible to suppress an impairment of the binding of the metal particle 13e to the second substance 13d bound to the detection target 13c, as well as to reduce a decrease in the rate of binding reaction between the second substance 13d and the metal particle 13e.

As shown in FIG. 6(c), the metal particle 13e can be supplied to the base 10 in a state of being contained in a second solution 13L2 which differs from the first solution 13L1. In this case, in contrast to a case where the metal particle 13e is put in a predetermined solution together with the second substance 13d, it is possible to select an optimum solution for the metal particle 13e, and thereby, for example, restrain metal particle 13e agglomeration, and also provide enhanced bindability between the metal particle 13e and the second substance 13d, even if the metal particle concentration is high. In consequence, even when the analyte liquid has a low content of the detection target 13c (contains the detection target 13c at low concentration), the binding of the second substance 13d and the metal particle 13e to the detection target 13c makes possible detection with good sensitivity.

As the second solution 13L2, for example, a solution similar to the first solution 13L1 can be used. The second solution 13L2 may be made larger in surfactant concentration than the first solution 13L1, or, a dispersant such as polyethylene glycol or polyvinyl methyl ether may be contained in the second solution 13L2. This makes it possible to restrain metal particle 13e agglomeration effectively.

(Step of Supplying Second Wash Solution 13W2)

Next, as shown in FIG. 7(b), after supplying the metal particle 13e, a second wash solution 13W2 may be supplied to the base 10.

In this step, for example, the metal particle 13e which remains unbound to the second substance 13d can be removed from the base 10 and vicinal areas. In consequence, in the subsequent step of supplying a metal ion and a reducing agent, it is possible to reduce the likelihood of the metal ion and the reducing agent being bound to an unnecessary residual substance, and thereby allow the metal ion and the reducing agent to act efficiently on the metal particle 13e bound to the second substance 13d.

The second wash solution 13W2 may either be identical with or differ from the second solution 13L2. In a case where the second wash solution 13W2 differs from the second solution 13L2, for example, the second wash solution 13W2 may be made larger in surfactant concentration than the first solution 13L1, or may be prepared by adding a different additional surfactant to the second solution 13L2. This makes it possible to efficiently remove the metal particle 13e which remains unbound to the second substance 13d from the base 10.

(Step of Supplying Metal Ion and Reducing Agent)

Next, as shown in FIG. 6(d), after supplying the metal particle 13e, the base 10 is supplied with a metal ion and a reducing agent for reduction of the metal ion.

In this step, on the surface of the metal particle 13e, the metal ion is reduced by the reducing agent, thus causing metal deposition on the surface of the metal particle 13e. In consequence, with respect to the weight of the metal particle 13e, the weight of the metal particle 13e having a deposited metal on a surface thereof becomes larger, which makes possible detection of the detection target 13c with good sensitivity.

Examples of the metal ion include Au³⁺, Ag⁺, Cu²⁺, Zn²⁺, and Ni⁺. Moreover, as the reducing agent, it is possible to use any of inorganic and organic reducing agents which are capable of metal ion reduction, for example, hydroxyl amine, citric acid, iron sulfate, and ascorbic acid. When adopting Au³⁺ for use as the metal ion, it is advisable to use hydroxyl amine or citric acid for the reducing agent, and, when adopting Ag⁺ for use as the metal ion, iron sulfate can be used for the reducing agent.

(Detection of Detection Target 13c using Detecting Element 3)

In the case of performing, after the completion of such a sequence of process steps, detection of the detection target 13c contained in the analyte liquid with use of the SAW-utilizing detecting element 3 of the above-described sensor apparatus 100, a predetermined voltage from an external measuring apparatus is applied to the first IDT electrode 11 via the wiring line 7, the first extraction electrode 19, and so forth.

A part of the surface of the element substrate 10 which is formed with the first IDT electrode 11 is thereupon excited so as to produce SAW having a predetermined frequency. Part of the thereby produced SAW propagates toward the detecting section 13, passes through the detecting section 13, and reaches the second IDT electrode 12.

At this time, in the detecting section 13, the second substance 13d and the metal particle 13e are successively bound in the order named to the detection target 13c, and also the surface of the metal particle 13e is deposited with metal, and hence, by comparison with its own weight, the detection target 13c gains weight as the result of addition of the weights of the second substance 13d, the metal particle 13e, and the deposited metal 13f, wherefore the SAW passing under the detecting section 13 undergoes variations in characteristics such as phase correspondingly. In response to the arrival of the SAW having varied characteristics at the second IDT electrode 12, a corresponding voltage is developed in the second IDT electrode 12. Output of this voltage is produced via the second extraction electrode 20, the wiring line 7, and so forth, and, reading on the output is taken by an external measuring apparatus for measurement on the detection target 13c.

As described heretofore, in the detection target sensing method according to the embodiment, after supplying the detection target 13c to the base 10 having the first substance 13b3 immobilized on a surface thereof, the base 10 is supplied with the second substance 13d which is bindable to the detection target 13c, and hence, with the detection target 13c kept bound efficiently to the first substance 13b3, the second substance 13d can be bound efficiently to the detection target 13c. Under this condition, further binding of the metal particle 13e to the second substance 13d makes possible detection of the detection target 13c with better sensitivity.

(Step of Supplying Third Substance 13g)

As shown in FIG. 8(b), after supplying the second substance 13d, the base 10 may be supplied with a third substance 13g which is bindable to the second substance 13d. In this case, as shown in FIG. 8(b), the third substance 13g may be supplied in a state of being kept bound to the metal particle 13e.

Examples of the third substance 13g include antibody, nucleic acid, protein A, protein G, and sugar chain. For example, in a case where the second substance 13d is antibody, it is possible to use an antibody corresponding to the antibody used for the second substance 13d. On the other hand, in a case where the second substance 13d is nucleic acid, it is possible to use a nucleic acid having a sequence in complementary relation to part of the nucleic acid used for the second substance 13d. Note that the third substance 13g has the same role as the above-described linker 13L when formed of a material which is bindable to the metal particle 13e.

Moreover, the third substance 13g can be used in combination with the linker 13L. With the combined use of the third substance 13g and the linker 13L, the third substance 13g and the linker 13L effect the binding of the metal particle in conjunction with each other, and hence, as compared with a case where the third substance 13g and the linker 13L are used separately, a greater number of metal particles can be bound to the detection target 13c. This makes possible detection of the detection target 13c with even better sensitivity.

### MODIFIED EXAMPLES

As modified examples of the detection target sensing method in accordance with the embodiment of the invention thus far described, as shown in FIG. 8(c), a blocking substance 13B may be bound to at least one of the surface of the base 10 and the surface of the metal particle 13e.

The blocking substance 13B bound to the surface of the base 10 serves to reduce or suppress the binding of the detection target 13c, the second substance 13d, and the metal particle 13e to the base 10. As the blocking substance 13B, it is possible to use a substance which will not hinder the binding of the detection target 13c to the first substance 13b3, the binding of the second substance 13d to the detection target 13c, and the binding of the metal particle 13e to the second substance 13d. On the other hand, the blocking substance 13B bound to the surface of the metal particle 13e serves to reduce or suppress the binding of the metal particle 13e to a substance other than the second substance 13d, and, in this case, it is possible to use a substance which will not hinder the binding of the metal particle 13e to the second substance 13d.

In the case of binding the blocking substance 13B to the surface of the base 10, the binding may be effected before supplying the detection target 13c as shown in FIG. 6(a). Moreover, in the case of binding the blocking substance 13B to the surface of the metal particle 13e, the blocking substance 13B may be blended in the second solution 13L2 together with the metal particle 13e as shown in FIG. 6(c).

Examples of the blocking substance 13B include BSA (bovine serum albumin), whey protein, polyethylene glycol, MPC (methacryloyloxyethyl phosphorylcholine) polymer, betaine polymer, and HEMA (hydroxyethyl methacrylate) polymer. Moreover, the above-described organic member can be used in an as-is state for the blocking substance 13B.

The invention may be carried into effect in various forms without being limited to the embodiments thus far described.

For example, although the embodiments have been described with respect to the case where the detecting element 3 has two or less detecting sections 13, the design of the detecting element 3 is not limited to this, and hence, three or more detecting sections 13 may be provided. This makes possible measurement on a greater number of substances, and highly accurate measurement on any specific substance as well.

Moreover, although the embodiments have been described with respect to the case where the detecting section 13 comprises a metallic film and an aptamer immobilized on the surface of the metallic film, as described above, for example, the detecting section 13 may be defined by a region between the first IDT electrode 11 and the second IDT electrode 12 on the surface of the base 10 composed of a piezoelectric substrate without using the metallic film.

Moreover, although the sensor according to the embodiment has been illustrated as being exemplified by a SAW (Surface Acoustic Wave) sensor, for example, a measurement cell for use in measurement by an SPR (Surface Plasmon Resonance) apparatus, or a QCM (Quartz Crystal Microbalance) sensor may be adopted instead. For example, when using the detecting element 3 provided with an optical waveguide or the like for induction of surface plasmon resonance, for example, the sensor takes reading on variation in optical refractive index at the detecting section. Otherwise, when using the detecting element 3 composed of a piezoelectric substrate such as a quartz substrate provided with an oscillator, for example, the sensor takes reading on variation in oscillation frequency in the oscillator.

Moreover, for example, in constructing the detecting element 3, a plurality of different devices may be co-arranged on a single base 10. For example, an enzyme electrode for use with the enzyme electrode method may be disposed next to a SAW device. In this case, in addition to measurement based on the immunization method using antibody or aptamer, measurement based on the enzymatic method can be conducted, and it is possible to increase items which can be inspected at one time.

Moreover, for example, although the embodiments have been described with respect to the case where the first cover member 1 comprises the first upstream portion 1Aa and the first downstream portion 1Ab, and the second cover member 2 comprises the third substrate 2a and the fourth substrate 2b, the invention is not limited to this, and hence, from among the first upstream portion 1Aa, the first downstream portion 1Ab, the third substrate 2a, and the fourth substrate 2b, some may be combined into an unitary structure, and more specifically, for example, the first cover member 1 composed of a unitary structure of the first upstream portion 1Aa and the first downstream portion 1Ab may be used.

Moreover, a groove portion may be provided either in one of the first cover member 1 and the second cover member 2 or in each of them. For example, when providing the groove portion in each of the first cover member 1 and the second cover member 2, the flow channel 15 may be created by joining these members together while maintaining alignment between the two groove portions, whereas, when providing the groove portion in one of the first cover member 1 and the second cover member 2, the flow channel 15 may be created by joining these members together so that the groove portion of one of the members faces the surface of the other.

Moreover, for example, although the embodiments have been described with respect to the case where the analyte has a liquid form (analyte liquid), the analyte is not limited to this form. That is, the analyte is not limited to a liquid form in so far as it is measurable by the sensor according to the embodiment, but may be of, for example, a gel form or a gaseous form. Moreover, the analyte may be made changeable in its state, and more specifically, for example, it may be designed to undergo a transition from a liquid state to a solid state as it flows through the flow channel 15 (flows over the detecting section 13).

### REFERENCE SIGNS LIST

1: First cover member

1A: Intermediate cover member

1Aa: First upstream portion

1Ab: First downstream portion

2: Second cover member

2a: Third substrate

2b: Fourth substrate

3: Detecting element

4: Recess-forming area

5: Element receiving recess

6: Terminal

7: Wiring line

10: Element substrate (Base)

11: First IDT electrode

12: Second IDT electrode

13: Detecting section

13a: Immobilization film

13b: Reaction portion

13b3: First substance

13c: Detection target

13d: Second substance

13L1: First solution

13e: Metal particle

13L2: Second solution

13f: Deposited metal

13W1: First wash solution

13W2: Second wash solution

13L: Linker

13B: Blocking substance

13g: Third substance

14: Inlet port

15: Flow channel

18: Air release hole

19: First extraction electrode

19e: End (Pad portion)

20: Second extraction electrode

20e: End (Pad portion)

27: Lead wire (Metallic thin wire)

28: Insulating member

29: Element electrode

100: Sensor apparatus

1. A detection target sensing method, comprising:
supplying a detection target to a base having a first substance immobilized on a surface thereof, the detection target being bindable to the first substance;
supplying a second substance to the base after the detection target is supplied thereto, the second substance being bindable to the detection target; and
supplying a metal particle to the base after the second substance is supplied thereto, the metal particle being bindable to the second substance.

2. The detection target sensing method according to claim 1, wherein
the second substance is supplied in a state of being contained in a first solution, and
the metal particle is supplied in a state of being contained in a second solution which differs from the first solution.

3. The detection target sensing method according to claim 1, further comprising:
supplying a first wash solution to the base after supplying the second substance and before supplying the metal particle.

4. The detection target sensing method according to claim 1, further comprising:
supplying a second wash solution to the base after supplying the metal particle.

5. The detection target sensing method according to claim 1, further comprising:
supplying a metal ion and a reducing agent for reduction of the metal ion to the base after supplying the metal particle.

6. The detection target sensing method according to claim 1, further comprising:
supplying a linker to the base, the linker being bindable to the second substance and the metal particle.

7. The detection target sensing method according to claim 6, wherein the linker comprises a first linker which is bindable to the second substance.

8. The detection target sensing method according to claim 7, wherein the second substance is supplied in a state of being kept bound to the first linker.

9. The detection target sensing method according to claim 7, wherein the linker further comprises a second linker which is bindable to the metal particle.

10. The detection target sensing method according to claim 9, wherein the metal particle is supplied in a state of being kept bound to the second linker.

11. The detection target sensing method according to claim 6, wherein the linker contains streptavidin and biotin.

12. The detection target sensing method according to claim 6, wherein the second substance and the metal particle are bound to each other via streptavidin bound to the second substance and biotin bound to the metal particle.

13. The detection target sensing method according to claim 6, wherein the second substance and the metal particle are bound to each other via biotin bound to the second substance and streptavidin bound to the metal particle.

14. The detection target sensing method according to claim 6, wherein the linker is supplied after supplying the second substance and before supplying the metal particle.

15. The detection target sensing method according to claim 1, further comprising:
binding a blocking substance to at least one of a surface of the base and a surface of the metal particle.

16. The detection target sensing method according to claim 1, further comprising:
supplying a third substance which is bindable to the second substance to the base after supplying the second substance.

17. The detection target sensing method according to claim 16, wherein the third substance is supplied in a state of being kept bound to the metal particle.
